(19) United States
(12) Patent Application Publication
(10) Pub. No.: US 2006/0073119 A1
(43) Pub. Date: Apr. 6, 2006

(54) Methods for treating neurodegenerative disorders

(75) Inventors: John Forsayeth, San Francisco, CA (US); Laura Sanftner, El Sobrante, CA (US)

Correspondence Address: ROBINS & PASTERNAK LLP, 1731 EMBARCADERO ROAD, SUITE 230, PALO ALTO, CA, 94303, US

(73) Assignee: Avigen, Inc., Alameda, CA (US)

(21) Appl. No.: 11/218,342

(22) Filed: Sep. 1, 2005

Related U.S. Application Data
(60) Provisional application No. 60/606,734, filed on Sep. 1, 2004.

(51) Int. Cl. A61K 48/00; C12N 15/861

(52) U.S. Cl. 424/93.2; 435/456

(57) ABSTRACT

Vectors and methods are provided for treatment of neurodegenerative disorders by administration of anti-inflammatory cytokines, such as IL-10. Anti-inflammatory cytokines can be administered as a protein or by gene therapy, using plasmid delivery or a viral vector such as adeno-associated virus (AAV). Diseases including Parkinson's disease, Amyotrophic Lateral Sclerosis, Alzheimer's disease and Multiple Sclerosis may be treated using the vectors and methods of the invention.

### CROSS-REFERENCE To RELATED APPLICATION

This application claims the benefit under 35 U.S.C. §119(e)(1) of U.S. application Ser. No. 60/606,734, filed Sep. 1, 2004, which application is incorporated herein by reference in its entirety.

### FIELD OF THE INVENTION

The present invention relates to treatment of neurodegenerative disorders by delivering an anti-inflammatory cytokine to the central nervous system (CNS). The invention encompasses both direct administration of an anti-inflammatory cytokine and delivery of a gene encoding the anti-inflammatory cytokine by gene therapy using an adeno-associated virus (AAV) vector.

### BACKGROUND OF THE INVENTION

Neurodegenerative disorders, such as Parkinson's disease (PD), amyotrophic lateral sclerosis (ALS), Alzheimer's disease (AD) and multiple sclerosis (MS) will become increasingly prevalent in the United States as the population ages. The need exists for improved methods of treatment of such neurodegenerative diseases.

PD is a neurodegenerative disease marked by the death of neurons in the substantia nigra region of the brain, leading to decreased production of the neurotransmitter dopamine. Symptoms begin to occur after the loss of approximately 80% of dopamine producing neurons in the substantia nigra. Typical symptoms include tremor, bradykinesia, rigidity and postural instability. It is estimated that 1.5 million American suffer from PD and that 60,000 new cases are diagnosed annually.

MS is an autoimmune disease in which the myelin sheath surrounding the CNS (including the brain, spinal cord and optic nerve) is damaged. During periods of MS activity, T-cells initiate an inflammatory response in myelinated tissue in the CNS, inducing demyelination of axons and axon loss. This inflammation can also cause killing of glial cells in the region of MS lesions. About 400,000 people currently suffer from MS in the United States, and about 10,000 news cases are diagnosed each year.

AD is the most common form of dementia among older people, with an estimated 4.5 million cases in the United States alone. In AD, nerve cells die in the area of the brain vital to memory and other mental abilities. The disease is characterized by accumulation of amyloid plaques and neurofibrillary tangles.

ALS is a relentlessly progressive lethal neurodegenerative disease involving selective annihilation of motor neurons. It is estimated that 30,000 people in the United States have ALS, and that 5600 people are diagnosed with the disease each year. Mutations in a gene encoding superoxide dismutase (SOD1) have been associated with approximately 20% of familial ALS (fALS) cases. Julien (2001) Cell 104:581-91. Further information relating to ALS can be found in the Online Mendelian Inheritance in Man (OMIM) entry # 105400, and in Rowland and Shneider (2001) New Eng. J. Med. 344:1688-1700, the disclosures of which are hereby incorporated by reference in their entireties. Transgenic mice overexpressing the mutant SOD1 gene, in which glycine 93 has been mutated to alanine (G93A), develop a dominantly inherited adult-onset paralytic disorder that has many of the clinical and pathological features of fALS. Gurney et al. (1994) Science 264:1772-75. However, to date, the molecular mechanisms leading to motoneuron degeneration in ALS and most motor neuron diseases remain poorly understood, and there is currently no therapy available to prevent or cure ALS.

The need exists for improved methods of treatment for these and other neurodegenerative disorders.

### SUMMARY OF THE INVENTION

The present invention provides methods for treating neurodegenerative disorders and diseases in a subject, for example Parkinson's disease, by delivering an anti-inflammatory cytokine to the to the central nervous system (CNS) of the subject.

In one embodiment the anti-inflammatory cytokine itself is delivered to the CNS to achieve a therapeutic level of the cytokine in the CNS of the subject.

In another embodiment the anti-inflammatory cytokine is delivered using a preparation of a recombinant AAV (rAAV) vectors, e.g. plasmids or virions, comprising a transgene encoding the cytokine. The vector transduces one or more cells in the CNS and causes expression of the anti-inflammatory cytokine by the transduced cells at a therapeutically effective level.

In one embodiment the transgene encodes interleukin-10 (IL-10), or an active fragment thereof.

In various embodiments the anti-inflammatory cytokine, or the vector encoding the anti-inflammatory cytokine, is administered to the subject intranasally, intrathecally, intraventricularly, to the dorsal root ganglion (DRG), or to the brain (e.g. the striatum or substantia nigra). In one embodiment delivery is effected using convection enhanced delivery (CED). CED can be conducted, for example, using either an osmotic pump or an infusion pump.

In another embodiment, the anti-inflammatory cytokine is delivered by transducing cells in vitro with a preparation of a recombinant AAV (rAAV) vector comprising a transgene encoding the cytokine, and subsequently administering the transduced cells into the CNS of the subject. The transduced cells produce the anti-inflammatory cytokine in the CNS at a therapeutically effective level. In one embodiment the cells to be transduced ex vivo are autologous.

In another aspect, the invention provides for methods for delivering recombinant AAV virions encoding an anti-inflammatory cytokine, or an active fragment thereof, to a subject having a CNS disorder. In one embodiment the CNS disorder is Parkinson's disease (PD), the rAAV virions are administered into the striatum or substantia nigra of the CNS, and the transgene sequence encodes IL-10 or an active fragment thereof.

In another aspect of the invention, recombinant AAV virions prepared using methods of the present invention may be used to introduce genetic material into animals or isolated animal (including human) cells for research purposes. For example, methods of the present invention may be used to transduce cells in an animal with rAAV virions encoding an anti-inflammatory cytokine to gather preclinical data, to screen for potential drug candidates, or to create an animal model of a human disease.

These and other embodiments of the subject invention will readily occur to those of ordinary skill in the art in view of the disclosure herein.

### DESCRIPTION OF THE DRAWINGS

FIGS. 1A, 1B and IC illustrate the steps taken to generate the pAAV4.6CMVIL-10 plasmid.

FIG. 2 is a graph of hIL-10 expression from a plasmid transfected into HeLa cells 24 hours post-transfection. Human embryonic kidney (HEK 293) cells were transfected (one day after plating) with 1 μg each of pAAV4.6CMVhIL-10 plasmid, pAAV4.6CMVhIL-10mut plasmid, or control pVmLacZ plasmid. Supernatants were collected 24 hours after transfection and measured for human IL-10 using an antibody-based detection method (Becton Dickenson ELISA system, Becton, Dickinson and Company, Franklin Lakes, N.J.). Expression was measured using a SpectraMax® 340 ELISA reader and data were analyzed using SoftMax® Pro 4.3LS (Molecular Devices, Sunnyvale, Calif.).

FIG. 3 is a graph of IL-10 expression from HeLa cells transfected with AAV-hIL-10 virions 24 hours after transfection. HeLa D7-4 cells were plated on 6-well transduced (one day after plating) with AAV-hIL-10 viral vector at MOIs ranging from 0 to 10,000. Expression was measured using a SpectraMax® 340 ELISA reader and data were analyzed using SoftMax® Pro 4.3LS (Molecular Devices, Sunnyvale, Calif.).

### DETAILED DESCRIPTION OF THE INVENTION

The present invention relates to novel methods for studying or treating neurodegenerative disorders or diseases using anti-inflammatory cytokines, including but not limited to IL-4, IL-10 and IL-13. In one aspect, gene therapy is used to delivery genes encoding the cytokine to the CNS of animals or animal cells for research or therapeutic purposes. In another aspect the anti-inflammatory cytokines are administered directly, as a protein preparation, to the CNS of animals or animal cells. In one embodiment the animal is a human, and the anti-inflammatory cytokine is provided for therapeutic purposes.

Cytokines are a diverse group of proteins that act as chemical messengers within the immune system. They play a crucial role in mediating inflammatory and immune responses and are now known to stimulate important metabolic and behavioral pathways by promoting communication between immune cells and nerve cells within the brain. Pro-inflammatory cytokines such as IL-1, IL-6, and TNF augment the immune response by activating T cells, B cells, or endothelial cells and stimulating hematopoiesis. Anti-inflammatory cytokines such as IL-4, IL-10, and IL-13 act as a counter-balance to dampen immune response through activities such as inhibition of cytokine synthesis and inhibition of T cell proliferation. IL-10, in particular, is synthesized in the central nervous system where it acts to promote survival of neurons and glial cells (resident immune cells in the brain) by blocking the effects of cytokines that trigger cell death, and by enhancing expression of cell survival signals. IL-10 also limits inflammation in the brain by reducing synthesis of pro-inflammatory cytokines, suppressing cytokine receptor expression, and inhibiting receptor activation. Activated glial cells make crucial contributions to brain inflammatory responses, and IL-10 is an important modulator of glial cell responses in the brain.

Therapeutic Indications

Parkinson's disease (PD) is a common neurodegenerative disorder characterized by the progressive loss of dopamine-producing neurons in an area of the brain known as the substantia nigra pars compacta. The loss of these neurons is associated with activation of glial cells (microglia), which then mediate deleterious events such as production of pro-inflammatory prostaglandin, cytokines, and pro-oxidant reactive species (oxidative stress is a key component of PD pathophysiology). Postmortem analysis implicates microglial involvement in many neurodegenerative disorders, including PD.

While classical theories on the etiology of PD have focused on intra-neuronal changes, it has been suggested that immune regulation is critical for neuronal homeostasis and survival in PD. Microglia are implicated as the effectors for the selective degeneration of dopaminergic neurons in a number of animal models of PD using diverse stimuli to mimic the disease (Smeyne et al. (2001) Glia 34:73-80; Sherer et al. (2003) Neurosci. Lett. 341:87-90; Hunot et al. (2003) Ann. Neurol. 53 suppl. 3:S49-60; Delgado et al. (2003) FASEB J. 17:944-46; Gao et al. (2002) J. Neurochem. 81:1285-97; Wu et al. (2002) J. Neurosci. 22:1763-71). Treatment of purified populations of human microglia with inflammatory agents caused increased cytokine release that was completely inhibited by IL-10 (Lee et al. (2002) J. Neurosci. 69:94-103). Similar results were seen using cultured rat microglia (Ledeboer et al (2002) Eur. J. Neurosci. 16:1175-85). These results suggest that a cascade of inflammatory processes plays a key role in the pathophysiology of PD.

Multiple Sclerosis (MS) is both a chronic inflammatory auto-immune disease and a chronic neurodegenerative disease. It is characterized by infiltrating T-cell and macrophages that invade via a dysfunctional blood-brain barrier and mount an auto-immune response in the spinal cord and brain parenchyma. MS patients generally have plaques that are made up of extensive regions of fiber tract demyelination. Activated microglia, T-cells, and macrophages secrete pro-inflammatory chemokines and cytokines that produce further recruitment of inflammatory cells, creating a toxic micro-environment that leads to eventual axonal destruction. T-cell activation is augmented and maintained by pro-inflammatory cytokines such as interleukin-1 and 6 (IL-1, IL-6) and Tumor Necrosis Factor alpha (TNFα) that bind to and stimulate T-cell receptors. These pro-inflammatory cytokines may be inhibited or attenuated by anti-inflammatory molecules or cytokines, such as interleukin-10 (IL-10), a regulatory anti-inflammatory cytokine that plays a critical role in preventing uncontrolled T-cell mediated tissue destruction. In addition, other immune response inhibition strategies have demonstrated efficacy in animal models of MS. See Weinberg et al. (1999) J. Immunol. 162: 1818-26; Furlan et al. (1999) J. Immunol. 163:2403-9; Furlan et al. (2001) Gene Ther. 8:13-9.

The most common cause of cognitive decline in the elderly is AD. The neuropathology of AD is characterized by amyloid plaques and neurofibrillary tangles. The inflammatory response that accompanies chronic neurodegeneration in AD is characterized by microglial activation. Upregulation of microglial antigens and synthesis of inflammatory mediators is associated with chronic neurodegeneration in AD. Akiyama et al. (2000) Neurobiol. Aging 21:383-421. In vitro studies show that amyloid is a potent activator of microglia (Meda et al. (1995) Nature 374(6523):647-50) and is neurotoxic when added to cultures.

ALS, also known as Lou Gehrig's disease, is an almost invariably fatal disorder manifested by a progressive loss of muscle caused by degeneration of the large motor neurons in the brainstem and spinal cord. The proliferation and activation of microglia are prominent histological features of sporadic ALS and a number of transgenic mouse models of ALS. The presence of activated microglia, IgG and its receptor for Fc portion (FcgammaRI), and T lymphocytes in the spinal cord of both patients with ALS and experimental animal models of motor neuron disease strongly suggest that immune-inflammatory factors may be actively involved in the disease process. Ramasubbu et al. (2003) IL-10, an Immunomodulatory Cytokine, Delays Onset in a Mouse Model of ALS, 14^{th} International Symposium on ALS/MND (2003). See also West et al. (2004) J. Neurochem. 91:133-43.

As discussed above, the present invention involves administration of anti-inflammatory cytokines, such as IL-10, which is a potent modulator of microglial responses in brain, in therapy for neurodegenerative diseases such as PD, MS, AD and ALS, each of which involves an inflammatory response that IL-10 may attenuate. Other disorders that may be treatable by IL-10, or other anti-inflammatory cytokines, include fatal familial insomnia, Rasmussen's encephalitis, Down's syndrome, Huntington's disease, Gerstmann-Straussler-Scheinker disease, tuberous sclerosis, neuronal ceroid lipofuscinosis, subacute sclerosing panencephalitis, Lyme disease, tse tse's disease (African Sleeping Sickness), HIV dementia, bovine spongiform encephalopathy (“mad cow” disease), Creutzfeldt Jacob disease, Herpes simplex encephalitis, Herpes Zoster cerebellitis, general paresis (syphilis), tuberculous meningitis, tuberculous encephalitis, optic neuritis, granulomatous angiitis, temporal arthritis, cerebral vasculitis, Spatz-Lindenberg's disease, methamphetamine-associated vasculitis, cocaine-associated vasculitis, traumatic brain injury, stroke, Lance-Adams syndrome, post-anoxic encephalopathy, radiation necrosis, limbic encephalitis, progressive supranuclear palsy, striatonigral degeneration, corticocobasal ganglionic degeneration, primary progressive aphasia, frontotemporal dementia associated with chromosome 17, spinal muscular atrophy, HIV-associated myelopathy, HTLV-1-associated myelopathy (Tropical Spastic Paraparesis), tabes dorsalis (syphilis), transverse myelitis, post-polio syndrome, spinal cord injury, radiation myelopathy, Charcot-Marie-Tooth, HIV-associated polyneuropathies, campylobacter-associated motor axonopathies, Guillain Barre Syndrome, chronic inflammatory demyelinating polyneuropathy, diabetic amyotrophy avulsion, phantom limb, complex regional pain syndrome, diabetic neuropathies, paraneoplastic neuropathies, myotonic dystrophy, HTLV-1-associated myopathy, trichinosis, inflammatory myopathies (polymyositis, inclusion body myositis, dermatomyositis), sickle cell disease, alpha-1-antitrypsin deficiency, tuberculosis, subacute bacterial endocarditis, chronic viral hepatitis, viral cardiomyopathy, Chaga's disease, malaria, Coxsackie B infection, macular degeneration, retinitis pigmentosa, vasculitis, inflammatory bowel disease, Crohn's disease, rheumatoid arthritis, bullous pemphigus, Churg-Strauss syndrome, myocardial infarction, toxic epidermal necrolysis, shock, type-1 diabetes, autoimmune thyroiditis, lymphoma, ovarian cancer, Lupus (systemic lupus erythematosus), asthma, progeria, sarcoidosis, type-2 diabetes and metabolic syndrome.

Anti-Inflammatory Cytokines and Variants Thereof

In addition to IL-10, other anti-inflammatory cytokines and variants (e.g. inflammatory cytokine antagonists) may be used to treat neurodegenerative diseases according to the methods of the present invention. Such cytokines and agents include interleukin-1 receptor antagonist (IL-1ra), interleukin-4 (IL-4), interleukin-13 (IL-13), tumor necrosis factor soluble receptor (TNFsr), α-MSH and transforming growth factor-beta 1 (TGF-β1). The native molecules, as well as fragments and analogs thereof that retain the ability to reduce the effects of neurological disease in any of the known models, including those described further herein, are intended for use with the present invention. Moreover, sequences derived from any of numerous species can be used with the present invention, depending on the animal to be treated.

Nucleotide and amino acid sequences of each of these anti-inflammatory cytokines from several animal species, and variants thereof, are well known. For example, IL-10 has been isolated from a number of animal and viral species. IL-10 for use herein includes IL-10 from any of these various species. Non-limiting examples of viral IL-10 include the IL-10 homologues isolated from the herpesviruses such as from Epstein-Barr virus (see, e.g., Moore et al. (1990) Science 248:1230-34; Hsu et al. (1990) Science 250:830-32; Suzuki et al. (1995) J. Exp. Med. 182:477-86), cytomegalovirus (see, e.g., Lockridge et al. (2000) Virol. 268:272-280; Kotenko et al. (2000) Proc. Natl. Acad. Sci. USA 97:1695-1700), and equine herpesvirus (see, e.g., Rode et al. (1993) Virus Genes 7:111-16), as well as the IL-10 homologue from the OrF virus (see, e.g., Imlach et al. (2002) J. Gen. Virol. 83:1049-58 and Fleming et al. (2000) Virus Genes 21:85-95). Representative, non-limiting examples of other IL-10 sequences for use with the present invention include the sequences described in NCBI accession numbers NM000572, U63015, AF418271, AF247603, AF247604, AF247606, AF247605, AY029171, UL16720 (all human sequences); NM012854, L02926, X60675 (rat); NM010548, AF307012, M37897, M84340 (all mouse sequences); U38200 (equine); U39569, AF060520 (feline sequences); U00799 (bovine); U11421, Z29362 (ovine sequences); L26031, L26029 (macaque sequences); AF294758 (monkey); U33843 (canine); AF088887, AF068058 (rabbit sequences); AF012909, AF120030 (woodchuck sequences); AF026277 (possum); AF097510 (guinea pig); U11767 (deer); L37781 (gerbil); AB107649 (llama and camel).

Non-limiting examples of IL-1ra sequences for use with the present invention include the sequences described in NCBI accession numbers NM173843, NM173842, NM173841, NM000577, AY196903, BC009745, AJ005835, X64532, M63099, X77090, X52015, M55646 (all human sequences); NM174357, AB005148 (bovine sequences); NM031167, S64082, M57525, M644044 (mouse sequences); D21832, 568977, M57526 (rabbit sequences); SEG AB045625S, M63101 (rat sequences); AF216526, AY026462 (canine sequences); U92482, D83714 (equine sequences); AB038268 (dolphin).

Non-limiting examples of IL-4 sequences for use with the present invention include the sequences described in NCBI accession numbers NM172348, AF395008, AB015021, X16710, A00076, M13982, NM000589 (all human sequences); BC027514, NM021283, AF352783, M25892 (mouse sequences); NM173921, AH003241, M84745, M77120 (bovine sequences); AY130260 (chimp); AF097321, L26027 (monkey); AY096800, AF172168, Z11897, M96845 (ovine sequences); AF035404, AF305617 (equine sequences); AF239917, AF187322, AF054833, AF104245 (canine sequences); X16058 (rat); AF046213 (hamster); L07081 (cervine); U39634, X87408 (feline); X68330, L12991 (porcine sequences); U34273 (goat); AB020732 (dolphin); L37779 (gerbil); AF068058, AF169169 (rabbit sequences); AB107648 (llama and camel).

Non-limiting examples of IL-13 sequences for use with the present invention include the sequences described in NCBI accession numbers NM002188, U10307, AF377331, X69079 (all human sequences); NM053828, L26913 (rat sequences); AF385626, AF385625 (porcine sequences); AF244915 (canine); NM174089 (bovine); AY244790 (monkey); NM008355 (mouse); AB107658 (camel); AB107650 (llama).

Non-limiting examples of TGF-β1 sequences for use with the present invention include the sequences described in NCBI accession numbers NM000660, BD0097505, BD0097504, BD0097503, BD0097502 (all human sequences); NM021578, X52498 (rat sequences); AJ009862, NM011577, BC013738, M57902 (mouse sequences); AF461808, X12373, M23703 (porcine sequences); AF175709, X99438 (equine sequences); X76916 (ovine); X60296 (hamster); L34956 (canine).

Non-limiting examples of alpha-MSH sequences for use with the present invention include the sequences described in NCBI accession number NM 000939 (human); NM17451 (bovine); NM 008895 (mouse); and M11346 (xenopus).

Non-limiting examples of TNF receptor sequences for use with the present invention include the sequences described in NCBI accession numbers X55313, M60275, M63121, NM152942, NM001242, NM152877, NM152876, NM152875, NM152874, NM152873, NM152872, NM152871, NM000043, NM 001065, NM001066, NM148974, NM148973, NM148972, NM148971, NM148970, NM148969, NM148968, NM148967, NM148966, NM148965, NM003790, NM032945, NM003823, NM001243, NM152854, NM001250 (all human sequences); NM013091, M651122 (rat sequences).

Polynucleotides encoding the desired anti-inflammatory cytokine for use with the present invention can be made using standard techniques of molecular biology. For example, polynucleotide sequences coding for the above-described molecules can be obtained by screening cDNA libraries from cells expressing the gene, or from genomic libraries, or by deriving the gene from a vector known to include the desired gene. Desired sequences may also be obtained by polymerase chain reaction (PCR) of any of the aforementioned libraries or vectors, using primers with sequences that selectively amplify the sequence of interest.

The gene of interest can also be produced synthetically based on the known sequences. Overlapping oligonucleotides encompassing the entire sequence of interest are prepared by standard methods and assembled into a complete coding sequence. See, e.g., Edge (1981) Nature 292:756; Nambair et al. (1984) Science 223:1299; Jay et al. (1984) J. Biol. Chem. 259:6311; Jayaraman et al. (1991) Proc. Natl. Acad. Sci. USA 88:4084-88. Additionally, oligonucleotide-directed synthesis (Jones et al. (1986) Nature 54:75-82), oligonucleotide directed mutagenesis of preexisting nucleotide regions (Riechmann et al. (1988) Nature 332:323-27 and Verhoeyen et al. (1988) Science 239:1534-36), and enzymatic filling-in of gapped oligonucleotides using T₄ DNA polymerase (Queen et al. (1989) Proc. Natl. Acad. Sci. USA 86:10029-33) can be used to provide molecules for use in the subject methods.

Delivery

Modes of delivery of an anti-inflammatory cytokine according to the present invention include delivery of the cytokine itself (as a protein) or administration of an AAV vector encoding the gene for the cytokine, either in vivo or ex vivo. Each mode of delivery has advantages over the other, and will be preferred in certain clinical settings.

Direct administration of the protein requires production of therapeutic amounts of the protein and repeated delivery to the CNS to achieve therapeutically effective levels. Large scale production of cytokines for therapeutic uses is well understood in the art and several cytokines are already approved for human therapeutic uses. Administration of a protein typically provides only transient efficacy, requiring frequent dosing, for example multiple administrations per day, often by intravenous injection. This transience, however, can be advantageous in many situations. For example, a subject with a traumatic injury to CNS tissue may be helped by transient cytokine therapy until the traumatic injury is resolved, at which point cytokine therapy may be discontinued. In addition, transient therapy may be discontinued relatively suddenly by simply withholding further doses, or dosing may be modified (e.g. in response to observed clinical effects) simply by changing the dosing of successive administrations. In contrast, gene therapy, particularly gene therapy lacking regulatable protein expression, may provide undesirable long term expression of a transgene long after it is needed, and the level of cytokine production can be difficult to regulate.

Gene therapy, on the other hand, has the advantage of potentially long-term therapeutic benefit with only one, or perhaps a limited number, of administrations. These methods allow clinicians to introduce DNA coding for a gene of interest directly into a patient (in vivo gene therapy) or into cells isolated from a patient or a donor (ex vivo gene therapy). Therapeutic proteins produced by transduced cells after gene therapy may be maintained at a relatively constant level in the CNS of a subject, as compared to a protein that is administered directly, which will typically vary greatly in concentration between the time right after administration of a first dose and the time immediately before the succeeding dose. Such sustained production of a therapeutic cytokine is particularly appropriate in the treatment of chronic diseases, such as neurodegenerative diseases. In addition, because gene therapy may require only a single administration, it is possible to use highly invasive procedures that would not be practical for the repeated administrations of protein, such as stereotactic intra-cranial injection.

Further, regulatable genetic constructs using small molecule inducers have been developed that might be included in vectors to be used in gene therapy embodiments of the present invention. Rivera et al. (1996) Nat. Med. 2:1028-32; No et al. (1996) Proc. Natl. Acad. Sci. USA, 93:3346-51; Gossen and Bujard (1992) Proc. Natl. Acad. Sci. USA 89:5547-51; the GeneSwitch® system (Valentis, Inc., Burlingame, Calif.). These systems are based on the use of engineered transcription factors whose activity is controlled by a small molecule drug, and a transgene whose expression is driven by the regulated transcription factor. One such system, based on induction by rapamycin (referred to herein as the “dimerizer system”), involves formation of a functional transcription factor from two synthetic fusion proteins dependent upon addition of rapamycin. Rivera et al. (1996) Nat. Med. 2:1028-32; Pollock et al. (2000) Proc. Natl. Acad. Sci. USA 97:13221-26. The dimerizer system is a component of the ARGENT Transcription Technology platform of ARIAD Pharmaceuticals, Inc. (Cambridge, Mass.). See U.S. Pat. Nos. 6,043,082 and 6,649,595; Rivera et al. (1999) Proc. Natl. Acad. Sci. USA 96:8657-62.

Gene Therapy

DNA may be introduced into a patient's cells in several ways. There are transfection methods, including chemical methods such as calcium phosphate precipitation and liposome-mediated transfection, and physical methods such as electroporation. In general, transfection methods are not suitable for in vivo gene delivery. Genes can be delivered using “naked” DNA in plasmid form. There are also methods that use recombinant viruses. Current viral-mediated gene delivery methods employ retrovirus, adenovirus, herpes virus, pox virus, and adeno-associated virus (AAV) vectors. Of the more than one hundred gene therapy trials conducted, more than 95% used viral-mediated gene delivery. C. P. Hodgson, Bio/Technology 13, 222-225 (1995).

In general, as used herein, the term “vector” refers to any genetic element, such as a plasmid, phage, transposon, cosmid, chromosome, virus, virion, etc., that is capable of replication when associated with the proper control elements and that can transfer gene sequences to cells. Thus, the term includes cloning and expression vehicles, as well as viral vectors. By “recombinant vector” is meant a vector that includes a heterologous nucleic acid sequence, or “transgene,” that is capable of expression in vivo.

It may also be desirable to fuse the gene of interest to immunoglobulin molecules, for example the Fc portion of a mouse IgG2a with a noncytolytic mutation, to provide for sustained expression. Such a technique has been shown to provide for sustained expression of cytokines, such as IL-10, especially when combined with electroporation. See e.g. Jiang et al. (2003) J. Biochem. 133:423-27; Adachi et al. (2002) Gene Ther. 9:577-83.

It should be understood that more than one transgene can be expressed by the delivered recombinant vector. For example, the recombinant vectors can encode more than one anti-inflammatory cytokine. Alternatively, separate vectors, each expressing one or more different transgenes, can also be administered. Thus, multiple anti-inflammatory cytokines can be delivered concurrently or sequentially. Furthermore, it is also intended that the vectors delivered by the methods of the present invention be combined with other suitable compositions and therapies.

Plasmid-Directed Gene Delivery

Genes encoding an anti-inflammatory cytokine can be delivered using non-viral plasmid-based nucleic acid delivery systems, as described in U.S. Pat. Nos. 6,413,942, 6,214,804, 5,580,859, 5,589,466, 5,763,270 and 5,693,622, all incorporated herein by reference in their entireties. Plasmids will include the gene of interest operably linked to control elements that direct the expression of the gene in a target cell, which control elements are well known in the art. Plasmid DNA can be guided by a nuclear localization signal or like modification.

Alternatively, plasmid vectors encoding the gene of interest can be packaged in liposomes prior to delivery to a subject or to cells, as described in U.S. Pat. Nos. 5,580,859, 5,549,127, 5,264,618, 5,703,055, all incorporated herein by reference in their entireties. For a review of the use of liposomes as carriers for delivery of nucleic acids, see, Hug and Sleight (1991) Biochim. Biophys. Acta. 1097:1-17; Straubinger et al. (1983) in Methods of Enzymology Vol. 101, pp. 512-27; de Lima et al. (2003) Current Medicinal Chemistry, Volume 10(14): 1221-31. The DNA can also be delivered in cochleate lipid compositions similar to those described by Papahadjopoulos et al. (1975) Biochem. Biophys. Acta. 394:483-491. See also U.S. Pat. Nos. 4,663,161 and 4,871,488, incorporated herein by reference in their entireties. In one embodiment, the plasmid vector is complexed with Lipofectamine 2000 at a ratio of 3 μl of Lipofectamine per μg of DNA. Wang et al. (2005) Mol. Therapy 12(2):314-320.

Biolistic delivery systems employing particulate carriers such as gold and tungsten may also be used to deliver genes of interest. The particles are coated with the gene to be delivered and accelerated to high velocity, generally under reduced pressure, using a gun powder discharge from a “gene gun.” See, e.g., U.S. Pat. Nos. 4,945,050, 5,036,006, 5,100,792, 5,179,022, 5,371,015, and 5,478,744, all incorporated herein by reference in their entireties.

A wide variety of other methods can be used to deliver the vectors. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, peptoid delivery, or microinjection. See, e.g., Sambrook et al (1989) Molecular Cloning, A Laboratory Manual, Cold Spring Harbor Laboratories, New York, for a discussion of techniques for transforming cells of interest; and Felgner, P. L. (1990) Advanced Drug Delivery Reviews 5:163-87, for a review of delivery systems useful for gene transfer. Exemplary methods of delivering DNA using electroporation are described in U.S. Pat. Nos. 6,132,419; 6,451,002, 6,418,341, 6,233,483, U.S. Patent Publication No. 2002/0146831, and International Publication No. WO/0045823, all of which are incorporated herein by reference in their entireties.

Plasmid vectors may also be introduced directly into the CNS by intrathecal (IT) injection, as described herein in greater detail with regard to protein administration. Plasmid DNA can be complexed with cationic agents such as polyethyleneimine (PEI) or Lipofectamine 2000 to facilitate uptake. See, e.g., Wang et al. (2005) Mol. Therapy 12(2):314-320. In one embodiment, a plasmid vector encoding an anti-inflammatory cytokine is complexed with PEI (25 kDa, Sigma-Aldrich, San Diego, Calif.) in a 5% glucose solution at a N/P ratio of approximately 15, where N represents PEI nitrogen and P represents DNA phosphate. Based on results obtained with pain relieving medications, intrathecal delivery may be expected to significantly reduce the required dose of a plasmid vector, e.g. up to ten-fold when compared with intravenous delivery, although such results may not apply to IT delivery of DNA-based therapeutic agents.

Retroviral Gene Delivery

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

Adenoviral Gene Delivery

In one embodiment of the subject invention, a nucleotide sequence encoding an anti-inflammatory cytokine is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Adenoviral vectors have several advantages in gene therapy. They infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase.

Adenoviral vectors for use with the present invention are derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that include the human gene for the anti-inflammatory cytokine IL-10, as well as vectors that include the gene for the anti-inflammatory cytokine IL-1ra, under the control of the Rous Sarcoma Virus (RSV) promoter.

Other recombinant adenoviruses of various serotypes, and comprising different promoter systems, can be created by those skilled in the art. See, e.g., U.S. Pat. No. 6,306,652, incorporated herein by reference in its entirety.

Moreover, “minimal” adenovirus vectors as described in U.S. Pat. No. 6,306,652 will find use with the present invention. Such vectors retain at least a portion of the viral genome required for encapsidation (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system.

Other useful adenovirus-based vectors for delivery of anti-inflammatory cytokines include the “gutless” (helper-dependent) adenovirus in which the vast majority of the viral genome has been removed. Wu et al. (2001) Anesthes. 94:1119-32. Such “gutless” adenoviral vectors produce essentially no viral proteins, thus allowing gene therapy to persist for over a year after a single administration. Parks (2000) Clin. Genet. 58:1-11; Tsai et al. (2000) Curr. Opin. Mol. Ther. 2:515-23. In addition, removal of the viral genome creates space that can be used to insert control sequences that provide for regulation of transgene expression by systemically administered drugs (Burcin et al. (1999) Proc. Natl. Acad. Sci. USA 96:355-60), adding both safety and control of virally driven protein expression. These and other recombinant adenoviruses will find use with the present methods.

Adeno Associated Virus (AAV) Gene Delivery

One viral system that has been used for gene delivery is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions.

Recombinant AAV virions comprising an anti-inflammatory cytokine gene may be produced using a variety of art-recognized techniques. In one embodiment, an rAAV vector construct is packaged into rAAV virions in cells co-transfected with wild-type AAV and a helper virus, such as adenovirus. See, e.g., U.S. Pat. No. 5,139,941.

Alternatively, plasmids can be used to supply the necessary replicative functions from AAV and/or a helper virus. In one embodiment of the present invention, rAAV virions are produced using a plasmid to supply necessary AAV replicative functions (the “AAV helper functions”). See e.g., U.S. Pat. Nos. 5,622,856 and 5,139,941, both incorporated herein by reference in their entireties. In another embodiment, a triple transfection method is used to produce rAAV virions. The triple transfection method is described in detail in U.S. Pat. Nos. 6,001,650 and 6,004,797, which are incorporated by reference herein in their entireties. The triple transduction method is advantageous because it does not require the use of an infectious helper virus during rAAV production, enabling production of a stock of rAAV virions essentially free of contaminating helper virus. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations. Vectors and cell lines necessary for preparing helper virus-free rAAV stocks are commercially available as the AAV Helper-Free System (Catalog No. 240071) (Stratagene, La Jolla, Calif.).

The AAV helper function vector encodes AAV helper function sequences (i.e., rep and cap) that function in trans for productive rAAV replication and encapsidation. Preferably, the AAV helper function vector supports efficient rAAV virion production without generating any detectable replication competent AAV virions (i.e., AAV virions containing functional rep and cap genes). An example of such a vector, pHLP19, is described in U.S. Pat. No. 6,001,650. The rep and cap genes of the AAV helper function vector can be derived from any of the known AAV serotypes. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (the “accessory functions”). The accessory functions include those functions required for AAV replication, including, without limitation, genes involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used. See U.S. Pat. No. 6,004,797. This plasmid provides a complete set of adenovirus accessory functions for AAV vector production, but lacks the components necessary to form replication-competent adenovirus.

Unlike stocks of rAAV vectors prepared using infectious helper virus, stocks prepared using an accessory function vector (e.g. the triple transfection method) do not contain contaminating helper virus because no helper virus is added during rAAV production. Even after purification, for example by CsCl density gradient centrifugation, rAAV stocks prepared using helper virus still remain contaminated with some level of residual helper virus. When adenovirus is used as the helper virus in preparing a stock of rAAV virions, contaminating adenovirus can be inactivated by heating to temperatures of approximately 60° C. for 20 minutes or more. This treatment effectively inactivates only the helper virus since AAV is extremely heat stable, while the helper adenovirus is heat labile. Although heat inactivating of rAAV stocks may render much of the contaminating adenovirus non-infectious, it does not physically remove the helper virus proteins from the stock. Such contaminating viral protein can elicit undesired immune responses in subjects and are to be avoided if possible. Contaminating adenovirus particles and proteins in rAAV stocks can be avoided by use of the accessory function vectors disclosed herein.

Recombinant AAV Expression Vectors

Recombinant AAV expression vectors are constructed using standard techniques of molecular biology. rAAV vectors comprise a transgene of interest (e.g. a gene encoding an anti-inflammatory cytokine) flanked by AAV ITRs at both ends. rAAV vectors are also constructed to contain transcription control elements operably linked to the transgene sequence, including a transcriptional initiation region and a transcriptional termination region. The control elements are selected to be functional in a mammalian target cell.

The nucleotide sequences of AAV ITR regions are known. See, e.g., Kotin (1994) Human Gene Therapy 5:793-801; Berns “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable transgenes for delivery in AAV vectors will be less than about 5 kilobases (kb) in size. The selected polynucleotide sequence is operably linked to control elements that direct the transcription thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector harboring a transgene of interest bounded by AAV ITRs can be constructed by directly inserting the selected sequence(s) into an AAV genome that has had the major AAV open reading frames (“ORFs”) excised. Other portions of the AAV genome can also be deleted, so long as enough of the ITRs remain to provide replication and packaging functions. Such constructs can be designed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-96; Vincent et al. (1990) Vaccines 90 (Cold Spring Harbor Laboratory Press); Carter (1992) Current Opinion in Biotechnology 3:533-39; Muzyczka (1992) Current Topics in Microbiol. and Immunol. 158:97-129; Kotin (1994) Human Gene Therapy 5:793-801; Shelling and Smith (1994) Gene Therapy 1:165-69; and Zhou et al. (1994) J. Exp. Med. 179:1867-75.

AAV ITR-containing DNA fragments can be ligated at both ends of a selected transgene using standard techniques, such as those described in Sambrook et al., supra. For example, ligations can be accomplished in 20 mM Tris-Cl pH 7.5, 10 mM MgCl₂, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Suitable host cells for producing rAAV virions of the present invention from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with an rAAV virion. Cells from the stable human cell line, 293 (readily available through the American Type Culture Collection under Accession Number ATCC CRL1573) are preferred in the practice of the present invention. The human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Other Viral Vectors for Gene Delivery

Additional viral vectors useful for delivering the nucleic acid molecules of interest include those derived from the pox family of viruses, including vaccinia virus and avian poxvirus. By way of example, vaccinia virus recombinants expressing a gene of interest can be constructed as follows. DNA carrying the gene is inserted into an appropriate vector adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter and the gene into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can be used to deliver the genes. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors, can also be used for gene delivery. Michael et al. (1993) J. Biol. Chem. 268:6866-69 and Wagner et al. (1992) Proc. Natl. Acad. Sci. USA 89:6099-6103. Members of the Alphavirus genus, for example the Sindbis and Semliki Forest viruses, may also be used as viral vectors for delivering the anti-inflammatory cytokine gene. See, e.g., Dubensky et al. (1996) J. Virol. 70:508-19; WO 95/07995; WO 96/17072.

Administration of Compositions of Therapeutic Cytokines

As explained above, agents that act on pro-inflammatory cytokines, such as any of the anti-inflammatory cytokines and cytokine antagonists described herein, can be administered alone, without gene therapy, or in conjunction with gene therapy, to treat or prevent neurodegenerative disease. Thus, for example, one or more of IL-10 (including viral IL-10), IL-1ra, IL-4, IL-13, TNFsr, alpha-MSH, TGF-β1, cytokine antagonists and/or other agents that act on proinflammatory cytokines can be formulated into compositions and delivered to subjects prior to, concurrent with or subsequent to gene delivery of one or more of these agents. Alternatively, these agents can be delivered alone, without the gene therapy.

With regard to therapy by administration of therapeutic anti-inflammatory cytokines, compositions of such cytokines will comprise a therapeutically effective amount of the agent such that the symptoms of neurodegenerative disease are reduced or reversed. The compositions will also contain a pharmaceutically acceptable excipient, as described above with reference to recombinant vectors. The pharmaceutical compositions may comprise the agent or its pharmaceutically acceptable salt or hydrate as the active component.

The agents may be formulated into compositions for CNS or peripheral nervous system delivery, of for oral (including buccal and sub-lingual), rectal, nasal, topical, pulmonary, vaginal or parenteral (including intramuscular, intraarterial, intrathecal, subcutaneous and intravenous) administration or in a form suitable for administration by inhalation or insufflation. A preferred manner of administration is into neural tissue including, without limitation, into peripheral nerves, the retina, dorsal root ganglia, neuromuscular junction, as well as the CNS, e.g., to target spinal cord glial or striatum cells, using any of the techniques described above with reference to recombinant vectors.

Intrathecal administration overcomes the blood-brain barrier (BBB) by direct injection into the cerebrospinal fluid. Intrathecal administration is described in greater detail with reference to administration of gene therapy vectors, infra.

Intranasal delivery (IND) is a noninvasive alternative method of bypassing the BBB to deliver therapeutic agents to the brain and spinal cord, eliminating the need for systemic delivery and thereby reducing unwanted systemic side effects. IND works because of the unique connection between the nerves involved in sensing odors and the external environment. Delivery from the nose to the central nervous system takes place within minutes along both the olfactory and trigeminal neural pathways. Delivery occurs by an extracellular route and does not require that the drugs bind to any receptor or undergo axonal transport. Bulk flow through perivascular and hemangiolymphatic channels may also be involved in the movement of drugs from the nose to the brain and spinal cord. The precise mechanism of IND is not an important element of the invention.

IL-10 therapy, for example, is targeted to regions of neurodegeneration where the anti-inflammatory cytokines would be expected to have a therapeutic effect through modulation of activated glial cells, e.g. the substantia nigra or the striatum in Parkinson's disease subjects. In other embodiments, therapy for MS, AD and ALS is intrathecally targeted.

In some embodiments, delivery of IL-10 to regions of the central nervous system is effected by convection enhanced delivery, as described in U.S. Pat. No. 6,309,634, incorporated herein by reference in its entirety, or by direct injection or other methods of infusion. In other embodiments delivery is accomplished by methods that incorporate systemic delivery and/or materials that facilitate crossing the blood-brain barrier. Preferably, the compositions are formulated in order to improve stability and extend the half-life of the active agent. For example, the active agent, such as IL-10, can be derivatized with polyethlene glycol (PEG). Pegylation techniques are well known in the art and include, for example, site-specific pegylation (see, e.g., Yamamoto et al. (2003) Nat. Biotech. 21:546-52; Manjula et al. (2003) Bioconjug. Chem. 14:464-72; Goodson and Katre (1990) Biotechnology 8:343-46; U.S. Pat. No. 6,310,180, all incorporated herein by reference in their entireties), pegylation using size exclusion reaction chromatography (see, e.g., Fee, C. J. (2003) Biotechnol. Bioeng. 82:200-06), and pegylation using solid phase (see, e.g., Lu and Felix (1993) Pept. Res. 6:140-46). For other methods of pegylation see, e.g., U.S. Pat. Nos. 5,206,344 and 6,423,685, incorporated herein by reference in their entireties, as well as reviews by Harris and Chess (2003) Nat. Rev. Drug. Discov. 2:214-21; Greenwald et al. (2003) Adv. Drug. Deliv. Rev. 55:217-56; and Delgado et al. (1992) Crit. Rev. Ther. Drug Carrier Syst. 9:249-304.

Moreover, the active agent may be fused to antibodies or peptides to improve stability and extend half-life using techniques well known in the art. For example, the active agent may be fused to immunoglobulin molecules in order to provide for sustained release. One convenient technique is to fuse the agent of interest to the Fc portion of a mouse IgG2a having a noncytolytic mutation. See, e.g., Jiang et al. (2003) J. Biochem. 133:423-27; and Adachi et al. (2002) Gene Ther. 9:577-83. Other methods for stabilizing the agent of interest are designed to make the protein larger or less accessible to proteases, such as by introducing glycosylation sites and/or removing sites involved in activation (e.g., that target the protein for degradation).

Additionally, the active agent may be delivered in sustained-release formulations. Controlled or sustained-release formulations are made by incorporating the protein into carriers or vehicles such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, the active agent can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al. (1993) Pharm. Res. 10:362-68; and McGee et al. (1997) J. Microencap. 14(2):197-210.

Administration of Compositions of Gene Therapy Vectors

Once produced, vectors or virions encoding the anti-inflammatory cytokine are formulated into compositions suitable for delivery. Compositions will comprise sufficient genetic material to produce a therapeutically effective amount of the anti-inflammatory cytokine of interest, i.e. an amount sufficient to reduce or ameliorate the symptoms of neurodegenerative disease. The compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Poloxamer (Pluronic F68), any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

One particularly useful formulation comprises the vector or virion of interest in combination with one or more dihydric or polyhydric alcohols, and, optionally, a detergent, such as a sorbitan ester. See, e.g., International Publication No. WO 00/32233.

Although representative doses of vector or virion are detailed below, one of skill in the art could determine an effective dose empirically. Methods of determining the most effective means and dosages of administration are well known to those of skill in the art and will vary with the vector, the composition of the therapy, the target cells, and the subject being treated. Administration can be effected in one dose, continuously or intermittently throughout the course of treatment. Single and multiple administrations can be carried out with the dose level and pattern being selected by the treating physician or researcher.

Recombinant vectors may be introduced into any neural tissue including, without limitation, peripheral nerves, retina, dorsal root ganglia, neuromuscular junction, as well as the CNS. Recombinant vectors of the present invention can be delivered using either ex vivo or in vivo transduction techniques.

For ex vivo delivery, the desired recipient cell is removed from the subject, transduced with rAAV virions in vitro, formulated into a pharmaceutical composition and reintroduced into the subject in one or more doses. In some embodiments, recipient cells harboring the DNA of interest are screened using conventional techniques such as Southern blots and/or PCR, or by using selectable markers, prior to reintroduction into the subject. Alternatively, syngeneic or xenogeneic cells can be used for ex vivo therapy, provided that they will not generate an undesired immune response in the subject. Neural progenitor cells may also be transduced in vitro and then delivered to the CNS.

For in vivo delivery, recombinant vectors are formulated into pharmaceutical compositions and one or more doses are administered. Therapeutically effective doses can be readily determined by one of skill in the art and will depend on the particular delivery system used. For AAV-delivered anti-inflammatory cytokines, a therapeutically effective dose will include on the order of from about 10⁶ to 10¹⁵ of the rAAV virions, more preferably 10⁷ to 10¹², and even more preferably about 10⁸ to 10¹¹ of the rAAV virions (or viral genomes, also termed “vg”) per subject, or any value within these ranges. For adenovirus-delivered anti-inflammatory cytokines, a therapeutically effective dose will include about 10⁶ to 10¹² plaque forming units (PFU), preferably about 10⁷ to 10¹⁰ PFU, or any dose within these ranges that alleviates the symptoms of neurodegenerative disease.

Generally, from 1 μl to 1 ml of composition will be delivered, such as from 0.01 to about 0.5 ml, for example about 0.05 to about 0.3 ml, such as 0.08, 0.09, 0.1, 0.2 ml, etc., and any number within these ranges.

Recombinant vectors, or cells transduced in vitro, may be delivered directly to neural tissue by injection into the ventricular region, the striatum (e.g., the caudate nucleus or putamen of the striatum), the spinal cord or a neuromuscular junction with a needle, catheter or related device, using neurosurgical techniques known in the art, such as, where appropriate, by stereotactic injection. See, e.g., Stein et al. (1999) J. Virol. 73:3424-29; Davidson et al. (2000) Proc. Natl. Acad. Sci. (USA) 97:3428-32; Davidson et al. (1993) Nat. Genet. 3:219-23; and Alisky and Davidson (2000) Hum. Gene Ther. 11:2315-29.

One method for targeting the CNS is by intrathecal delivery. Intrathecal delivery is effected by delivering a therapeutic substance to the cerebrospinal fluid (CSF) in the intrathecal (subarachnoid) space, located between the arachnoid membrane and the pia mater, which adheres to the surface of the spinal cord and brain. Delivery to the intrathecal space bypasses the blood brain barrier (BBB), allowing for accumulation of a therapeutic substance within the CNS. The BBB also serves to prevent leaking of relatively impermeable substances (e.g. IL-10) into general circulation, thus avoiding systemic side effects that might otherwise occur.

Intrathecal injection is typically made at either the L3/L4 or L4/L5 intervertebral space in adult human subjects, or L4/5 or L5/S1 for infants. Because post-administration complications such as headache are associated with larger bore needles for intrathecal delivery, a small bore needle should be used, e.g. a 22-25 gauge pencil-point needle, e.g. Whitacre G27 (Becton-Dickinson, Rutherford, N.J.). Intrathecal delivery can be via bolus injection, which can optionally be repeated, or by continuous infusion using a surgically implanted catheter and pump (e.g. an osmotic pump). Commercially available systems for intrathecal delivery include the SynchroMed® EL and SynchroMed® II intrathecal drug delivery systems (Medtronic, Minneapolis, Minn.). The details of intrathecal administration procedure, however, will be determined by a researcher or medical practitioner in light of the subject at issue, and is not a crucial aspect of the present invention.

Intrathecal delivery presents many advantages. Protein expressed from the rAAV vector is released into the surrounding CSF, and unlike viruses, released proteins can penetrate into the spinal cord parenchyma, just as they do after acute intrathecal injections. Indeed, intrathecal delivery of viral vectors can keep expression local. Moreover, in the case of IL-10, its brief half-life also serves to keep it local following intrathecal gene therapy; that is, its rapid degradation keeps the active protein concentrated close to its site of release. An additional advantage of intrathecal gene therapy is that the intrathecal route mimics lumbar puncture administration already in routine use in humans.

Another method for administering recombinant vectors or transduced cells is by delivery to dorsal root ganglia (DRG) neurons, e.g., by injection into the epidural space with subsequent diffusion to DRG. For example, recombinant vectors or transduced cells can be delivered via intrathecal cannulation under conditions where the protein diffuses to DRG. See Chiang (2000) Acta Anaesthesiol. Sin. 38:31-36; Jain (2000) Expert Opin. Investig. Drugs 9:2403-10.

Yet another mode of administration to the CNS uses convection-enhanced delivery (CED). Bobo et al. (1994) Proc. Nat'l Acad. Sci (USA) 91:2076-80. In this way, recombinant vectors can be delivered to many cells over large areas of the CNS. Moreover, the delivered vectors efficiently express transgenes in CNS cells (e.g., glial cells). Any convection-enhanced delivery device may be appropriate for delivery of recombinant vectors. In a preferred embodiment, the device is an osmotic pump or an infusion pump. Both osmotic and infusion pumps are commercially available from a variety of suppliers, for example Alzet Corporation, Hamilton Corporation, Alza, Inc. Typically, a recombinant vector is delivered via CED devices as follows. A catheter, cannula or other injection device is inserted into CNS tissue in the chosen subject. Stereotactic maps and positioning devices are available, for example from ASI Instruments (Warren, Mich.). Positioning may also be conducted by using anatomical maps obtained by CT and/or MRI imaging to help guide the injection device to the chosen target. Moreover, because the methods described herein can be practiced such that relatively large areas of the subject take up the recombinant vectors, fewer infusion cannula are needed. Since surgical complications are related to the number of penetrations, this mode of delivery serves to reduce the side-effects seen with conventional delivery techniques. For a detailed description regarding CED delivery, see U.S. Pat. No. 6,309,634, incorporated herein by reference in its entirety.

Gene therapy vectors may also be administered intranasally, or parenterally (including intramuscular, intraarterial, subcutaneous and intravenous). Intranasal administration is described in greater detail with reference to administration of gene therapy vectors, infra.

The following examples are offered for illustrative purposes only, and are not intended to limit the scope of the present invention in any way.

### EXAMPLE 1

### Plasmid-Directed Expression of Human IL-10 In Vitro

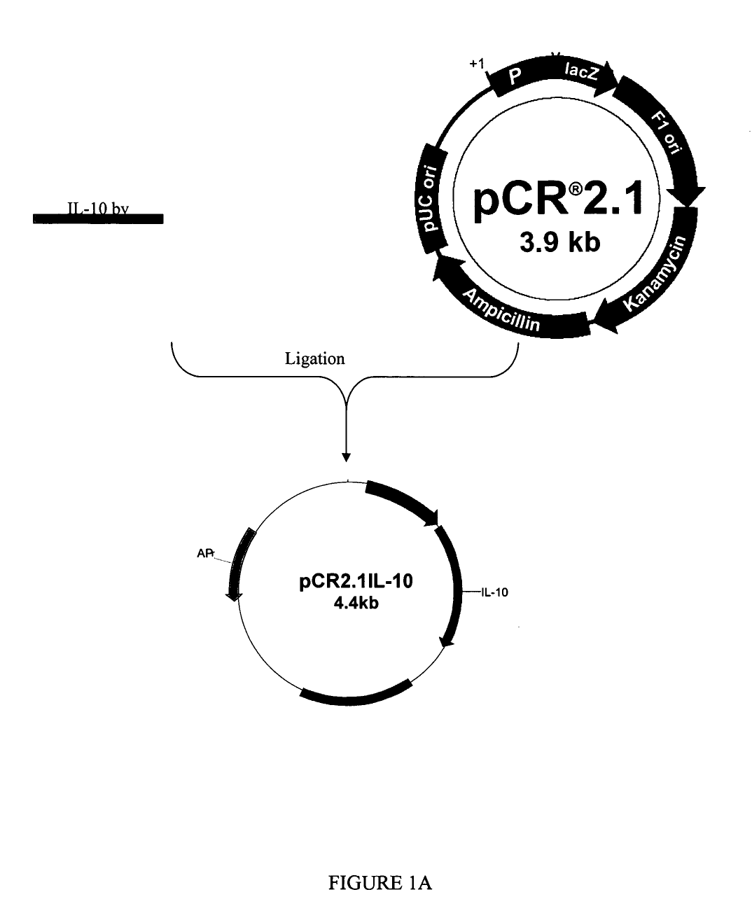

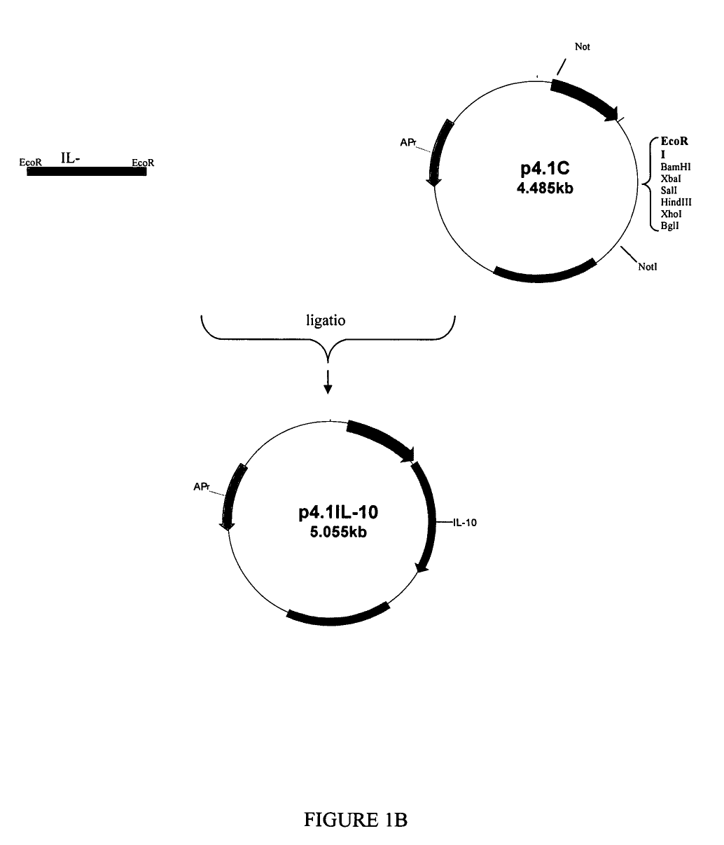

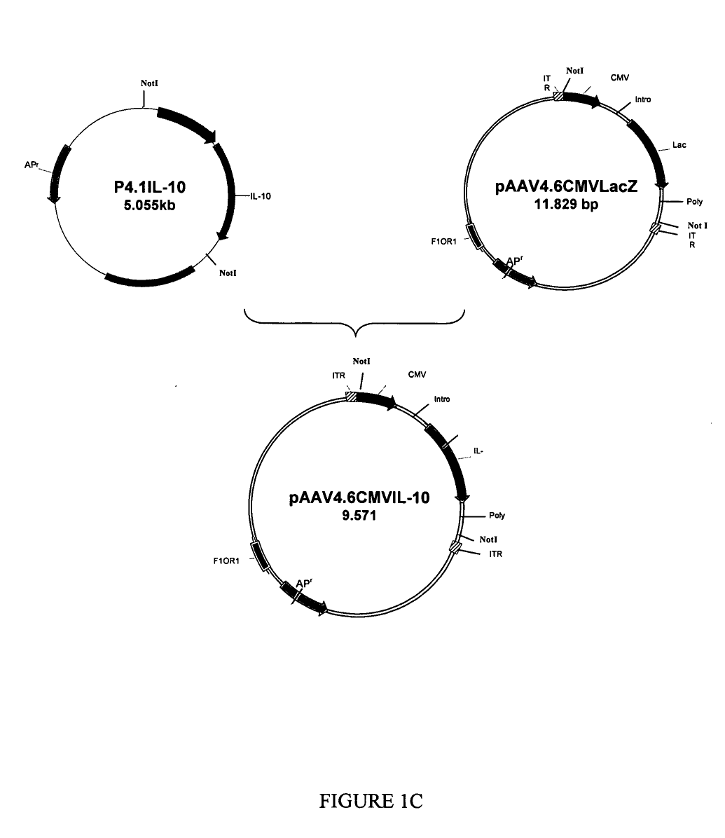

An AAV plasmid vector encoding human IL-10 is generated as follows. Human IL-10 (hIL-10) is amplified from human cDNA derived from leukocytes (Clontech, Mountain View, Calif.), using primers specific for the 5′ and 3′ ends, with the proof-reading polymerase Pfu under standard conditions. The resultant amplicon is subcloned into the pCR®2.1 shuffle vector to form pCR2.1 IL-10, illustrated in FIG. 1A. The confirmed human IL-10 cDNA is excised from the shuttle vector and inserted into a CMV promoter-driven expression cassette in the p4.1 c vector to form p4.1 IL-10, illustrated in FIG. 1B. The entire expression cassette (i.e. all sequence between the Not I sites including the CMV promoter, a chimeric CMV/beta-globin intron, human IL-10 cDNA, and the human growth hormone polyadenylation site) is subsequently inserted between flanking AAV inverted terminal repeats in the AAV transgene plasmid pAAV 4.6CMV LacZ (ITRs). FIG. 1C illustrates the resulting plasmid, referred to as pAAV4.6CMV IL-10.

A recombinant viral vector incorporating the sequence encoding human mutant L-10 (pAAV4.6CMV IL-10Mut) is constructed as described above, with the exception that site-specific mutagenesis is employed to replace isoleucine with alanine at position 87 in the protein. This single amino acid substitution has been reported to modulate IL-10 activity such that it retains its immunosuppressive characteristics but loses immunostimulatory properties (Ding et al. (2000) J. Exp. Med. 191: 213-23).

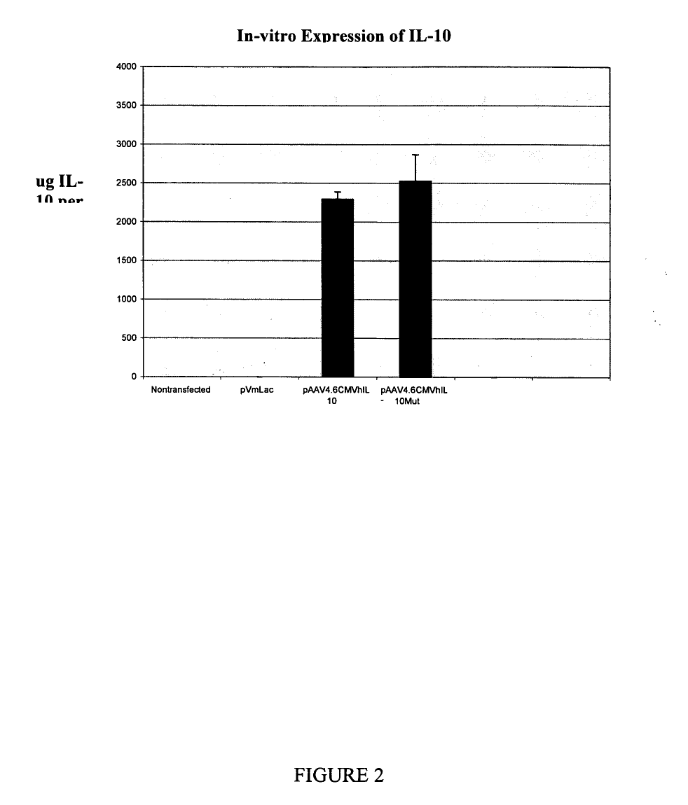

The plasmid constructs pAAV4.6CMV IL-10 and pAAV4.6CMV IL-10Mut are tested for their ability to direct IL-10 expression by transfection into HEK 293 cells and species-specific IL-10 ELISA. Results are presented in FIG. 2. No human IL-10 is detected in control samples that are not transfected, or are transfected with a plasmid directing expression of LacZ, whereas hIL-10 expression is observed with both IL-10 expression plasmids.

### EXAMPLE 2

### AAV-Directed Expression of Human IL-10 In Vitro

AAV-hIL-10 virions and AAV-hIL-10Mut virions are generated as follows. A derivative of human embryonic kidney 293 cells is transiently transfected with pAAV4.6CMV IL-10 (or the mutant form), which plasmids are described in Example 1. An accessory function plasmid (pladeno5) containing the E2A and E4 and VA RNAs genes from adenovirus type 2 is added, as well as pHLP19, an AAV helper plasmid that contains AAV rep and cap genes. Following transfection, cells are incubated for 2-3 days and collected. Harvested cells are concentrated by centrifugation and lysed using a freeze/thaw method to release AAV-IL-10 virions. Cellular debris is removed by centrifugation. The lysate supernatant is incubated in the presence of Benzonase® to reduce residual cellular and plasmid DNA. Calcium chloride is added to precipitate additional impurities, which are removed by centrifugation. Polyethylene glycol (PEG) is added to the clarified supernatant to selectively precipitate virions, which are collected by centrifugation. The virions are further purified by two cycles of isopycnic gradient ultracentrifugation in cesium chloride. Fractions containing AAV-hIL-10 are then pooled, and diafiltered using sterile phosphate buffered saline (PBS), pH 7.4, containing 5% sorbitol, by tangential flow filtration (TFF). Following recovery of the diafiltered virions from the TFF apparatus, Poloxamer 188 is added to a final concentration of 0.001%. The formulated virions are filtered (0.22 μm), aseptically dispensed into polypropylene cryovials, and labeled. The final product is stored frozen below −60° C.

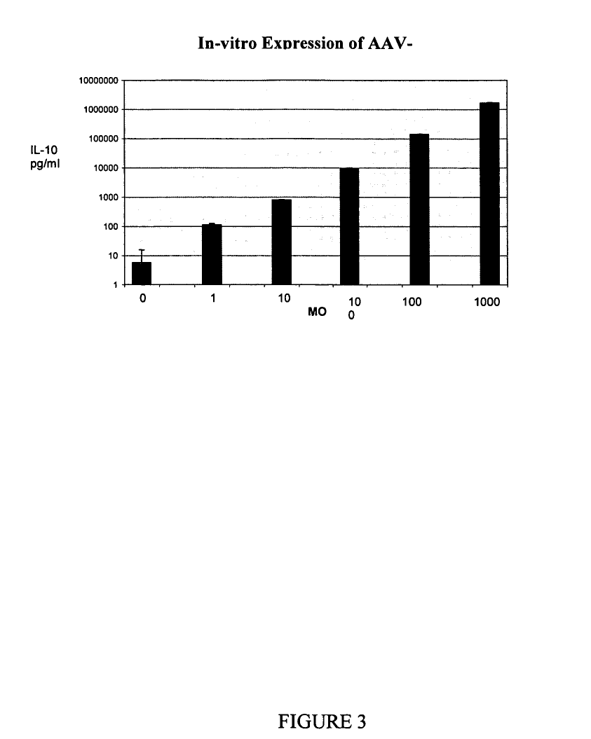

The ability of AAV-hIL-10 to direct transgene expression is confirmed by ELISA following transduction of HeLa cells with virion at several multiplicities-of-infections (MOIs), as illustrated in FIG. 3. IL-10 expression increases in a dose responsive manner as a function of MOI over the range studied (1-10,000).

### EXAMPLE 3

### AAV-Directed Expression of Human IL-10 in Rat Brain

AAV-hIL-10 virions are prepared as described in Examples 1 and 2. Virions are infused into the striatum of rats via convection enhanced stereotaxic delivery (5×10¹⁰ vg/hemisphere, in 5 μl). The human IL-10 transgene is expressed in the infused cells for up to 2 months, as measured by both hIL-10 immunohistochemistry and ELISA of homogenized tissue extracts (Table 1).

Expression of human IL-10 in rat brain is measured by human-specific IL-10 ELISA, with no rat IL-10 cross-reactivity, over a time course of 8 weeks. Animals are divided into three treatment groups: AAV-hIL-10-infused (8 rats, 16 hemispheres), excipient-infused (4 rats, 8 hemispheres), and naïve (4 rats, 8 hemispheres). The concentration of hIL-10 protein in striatal brain tissue is determined using a human IL-10 ELISA kit (KHC0102) (BioSource, Camarillo, Calif.) at 1, 2, 4, and 8 weeks post-infusion. At each time point two AAV-hIL-10-infused rats (4 hemispheres), one excipient-infused rat (2 hemispheres), and one naïve rat (2 hemispheres) are sacrificed.

The data in Table 1 demonstrate a rise in hIL-10 levels above background at 4 weeks post-infusion that is sustained at levels of 2,000-3,500 pg/ml for up to 8 weeks. The result is confirmed by immunohistochemistry of cross-sections of striatal tissue that has been immunohistochemically stained with anti-hIL-10 antibody at eight weeks post-infusion, viewed at 40× magnification. Whole brain cross-sections immunohistochemically stained for hIL-10 demonstrate expression of hIL-10 in the cytoplasm (green signal) of the medium spiny neurons of the striatum. Nuclei are counterstained with a nuclear stain, Dapi (blue).

TABLE 1
Expression of IL-10 in Rat Brain

| | Mean hIL-10 (pg/ml) in | | |
|---|---|---|---|
| | homogenized striatal tissue ± SD | | |
| Time | AAV-hIL- | Excipient- | |
| post-infusion | 10-infused | infused | Naïve |
| Week 1 | 30 ± 10 | 43 ± 2 | 65 ± 4 |
| Week 2 | 57 ± 14 | 62 ± 6 | 48 ± 4 |
| Week 4 | 3,498 ± 2,201 | 171 ± 21 | 20 ± 2 |
| Week 8 | 2,148 ± 863 | 79 ± 3 | 33 ± 2 |

### EXAMPLE 4

### IL-10 Gene Therapy in Rats Using AAV-IL-10

Rats are given 2.5×10¹⁰ particles of AAV-IL-10 using a Harvard infusion pump (Harvard Apparatus Inc., Holliston, Mass.) or Alzet subcutaneous osmotic pump (Alza Scientific Products, Palo Alto, Calif.). Female Sprague-Dawley rats (250-300 g) from Charles River Laboratories (Wilmington, Mass.) are anesthetized with an intraperitoneal injection of ketamine (100 mg/kg body weight) and xylazine (10 mg/kg body weight) and prepped for surgery. During surgery, sedation is maintained with isoflurane (Aerrane®, Ohmeda PPD Inc., Liberty, N.J.) and O₂ flow rates are kept at 0.3-0.5 L/min. The head of each rat is fixed in a stereotactic apparatus (Small Animal Stereotactic Frame; ASI Instruments, Warren, Mich.) with ear bars, and a midline incision is made through the skin to expose the cranium. A bore hole is made in the skull 1 mm anterior to the bregma and 2.6 mm lateral to the midline using a small dental drill. Virions are delivered to the left hemisphere and a depth of 5 mm using an infusion pump or subcutaneous osmotic pump.

AAV-IL-10 is continuously administered to each rat at a rate of 8 μl/h for 2.5 h using a Harvard infusion pump. The loading chamber (Teflon tubing 1/16th″ OD.times.0.03″ ID) and attached infusion chamber ( 1/16″ OD.times.0.02″ ID) are filled with 2.5×10⁸, 2.5×10⁹, or 2.5×10¹⁰ particles of AAV-IL-10 in a total volume of 20 μl. Delivery is effected through a 27 gauge needle fitted with fused silica, which is gradually removed 15 minutes following infusion.

Alternatively, subcutaneous osmotic pumps may be used to deliver vector. AAV-IL-10 is continuously administered to each rat at a rate of 8 μl/h for 24 h using Alzet osmotic pump model #2001D (ALZA Scientific Products, Palo Alto, Calif.). The pump's reservoir and attached catheter (polyethylene 60 tubing) are filled with 22.5×10¹⁰ particles of AAV-IL-10 in a total volume of 200 μl artificial CSF (Harvard Apparatus, Inc., Holliston, Mass.). Delivery is through a 27 gauge cannula fitted with fused silica. After stereotactic placement, the cannula is secured to the skull with a small stainless steel screw and dental cement, and the pump is implanted subcutaneously in the mid-scapular area of the back. The surgical site is closed in anatomical layers with 9 mm wound clips. Twenty four hours later, pumps are removed by clipping and sealing the catheters but the implanted cannulas are left in place. Burr holes are filled with bone wax.

### EXAMPLE 5

### AAV-IL-10 Gene Therapy in a Rat Model for Parkinson'S Disease

The ability of AAV-IL-10 gene therapy to treat Parkinson's disease is evaluated in an animal model of the disease as follows. Recombinant AAV virions carrying the sequence encoding the human IL-10 gene are created as described in Examples 1 and 2.

Rotational behavior is analyzed in unilaterally 6-hydroxydopamine (6-OHDA) lesioned rats both prior to and following convention enhanced delivery (CED) of recombinant AAV virions carrying a sequence encoding human IL-10. The 6-OHDA rat model has long been considered an appropriate model for studying Parkinson's disease. Acute challenge with dopamine-replacing drugs (such as L-dopa) or dopamine antagonists (such as apomorphine) elicits a rotational response in 6-OHDA-lesioned rats. This rotation is contraversive to the lesion and is considered an anti-parkinsonian effect.

Unilaterally lesioned rats to be used in the experiment are tested for rotational behavior prior to treatment with AAV-IL-10. Only rats exhibiting 160 or more rotations in 30 minutes are used in the experiment. All test rats must also exhibit robust contralateral rotation in response to apomorphine, and intramuscular administration of methyl-DOPA/benseroside (L-dopa) (5 mg/kg) must not induce rotational behavior.

Four such unilaterally lesioned rats are treated with AAV-IL-10 and another four are treated with a control recombinant AAV (e.g. AAV-GFP, a variant AAV carrying the gene for green fluorescent protein). AAV virion administration is as described in Example 4. Rotational behavior of both groups of rats in response to L-dopa administration is assessed two weeks after CED of AAV virions.

In vivo dopamine levels are measured in treated rats by microdialysis (Wang et al. (1994) Experimental Neurobiology 126:1-10). To inhibit catabolism of dopamine via the MaoB enzymatic pathway) paragyline (75 mg/kg) is administered to the rats intramuscularly prior to L-dopa administration.

### EXAMPLE 6

### IL-10 Delivery to Monkeys with MPTP-Induced Parkinson'S Disease

Rhesus monkeys (n=4, 3-5 kg) are chosen as candidates for implantation based on the evolution of their parkinsonian symptoms. Animals are lesioned by infusing 2.5-3.5 mg of 1-methyl-4-phenyl-1,2,3,4-tetrahydropyridine (MPTP)-HCl through the right internal carotid artery (referred to as ipsilateral side) followed by four I.V. doses of 0.3 mg/kg of MPTP-HCl until a stable, overlesioned hemi-parkinsonian syndrome is achieved (Eberling (1998) Brain Res. 805:259-62). MPTP is it converted in the CNS to MPP+ by monoamine oxidase B. MPP+ is a potent neurotoxin which causes degeneration of the nigral dopaminergic neurons and loss of the nigro-striatal dopamine pathway, as seen in Parkinson's disease. MPTP-lesioned animals are clinically evaluated once a week using a clinical rating scale and their activity is monitored for five months after lesioning before AAV vector is administered.

Following MPTP administration, the animals develop clinical signs of Parkinson's disease manifested by bradykinesia, rigidity, balance disturbances, and flexed posture. The left arm is less frequently used than the right and shows signs of tremor. Using the clinical rating scale, all of the monkeys have moderate to severe stable parkinsonian scores (e.g. 19-24) during the five month post-MPTP period.

AAV-hIL-10 virions are produced as described in Examples 1 and 2. Procedures for infusion of virions into the striatum of MPTP-treated monkeys are as follow.

Adult rhesus monkeys (n=4) are immobilized with a mixture of ketamine (Ketaset®, 10 mg/kg, intramuscular injection) and Valium® (0.5 mg/kg, intravenous injection), intubated and prepared for surgery. Isotonic fluids are delivered intravenously at 2 mL/kg/hr. Anesthesia is induced with isoflurane (Aerrane®, Omeda PPD, Inc., Liberty, N.J.) at 5% v/v, and then maintained at 1%-3% v/v for the duration of the surgery. The animal's head is placed in an MRI-compatible stereotaxic frame. Core temperature is maintained with a circulating water blanket while electrocardiogram, heart rate, oxygen saturation and body temperature are continuously monitored during the procedure. Burr-holes are made in the skull with a dental drill to expose areas of the dura just above the target sites.

AAV-hIL-10 is infused by CED (Lieberman et al. (1995) J. Neurosurg. 82(6):1021-29; Bankiewicz et al. (2000) Exp. Neurol. 164(1): 2-14). Each monkey receives a total of 3×10¹¹ vg in 200 μL spread over four sites (50 μL per site with two sites per hemisphere). Infusion cannulae are manually guided to the putamen in each brain hemisphere, and the animals receive bilateral infusions (i.e. sequential infusions to the rostral and caudal sites within both hemispheres) of AAV-hIL-10 (1.5×10¹² vg/mL) with either ramped infusion (0.2 μL/min (10 min), 0.5 μL/min (10 min), 0.8 μL/min (10 min) and 1 μL/min (35 min)) or a constant rate (1 μL/min (50 min)). Approximately 10 minutes after infusion, the cannulae are removed and the wound sites are closed. Animals are monitored for full recovery from anesthesia, placed in their home cages and clinically observed (twice a day) for approximately five days following surgery.

Following intrastriatal AAV administration, animals are assessed for any signs of abnormal behavior. Animals are observed and rated by veterinary technicians twice a day using clinical observation forms. Monkeys typically recover from the surgery within two hours and are able to maintain themselves, including feeding and grooming. There are typically no signs of any adverse effects during the 8-week post-surgical period.

Magnetic Resonance Imaging

Visualization of the target site is crucial for the precise placement of the infusion cannula within the caudate nucleus or putamen. Stereotactic procedures combined with MRI are used in order to accurately place the cannula within the desired targeted structures. All animals are scanned before surgery to generate accurate stereotactic coordinates of the target infusion sites for each individual animal. The same fiducial markers that are used for PET scanning are placed on the frame for co-registration of MRI and PET images. Briefly, during the scanning procedure, the animals are sedated using a mixture of ketamine (Ketaset, 7 mg/kg, im) and xylazine (Rompun®, 3 mg/kg, im). The animals are placed in an MRI-compatible stereotactic frame, earbar and eyebar measurements are recorded, and an IV line is established. Sixty coronal images (1 mm) and 15 sagittal images (3 mm) are taken using a GE Signa 1.5 Tesla machine. Magnetic resonance images are T1-weighted and obtained in three planes using a spoil grass sequence with a repetition time (TR)=700 ms, an echo time (TE)=20 ms and a flip angle of 30′. The field of view is 15 cm, with a 192 matrix and a 2 NEX (number of averages per signal information). Baseline scanning time is approximately 20 minutes. Rostro-caudal and medio-lateral distribution of a targeted structure (e.g., caudate nucleus) is determined using the coronal MR images. Surgical coordinates are determined from magnified coronal images (1.5×) of the caudate nucleus and putamen.

Positron Emission Tomography (PET)

All animals receive two PET scans, a baseline scan following establishment of the MPTP lesion, and a second scan 7-8 weeks after infusion with either AAV-hIL-10 or AAV-LacZ. Prior to PET, each animal undergoes magnetic resonance (MR) imaging using a 1.5 T magnet and a stereotaxic frame which permitted coregistration between PET and MR data sets through the use of external fiducial markers. The PET studies are performed on the PET-600 system, a singleslice tomograph with a resolution of 2.6 mm in-plane and an adjustable axial resolution which is increased from 6 mm to 3 mm for the current study by decreasing the shielding gap. The characteristics of this tomograph have been described previously (Budinger et al. (1991) Nucl. Med. Biol. 23(6):659-67; Valk (1990) Radiology 176(3):783-90). The monkeys are intubated and anesthetized with isoflurane, placed in a stereotaxic frame and positioned in the PET scanner so as to image a coronal brain slice passing through the striatum. Monkeys are positioned in the same way for each study using the anterior-posterior scales on the stereotaxic frame and a laser light connected to the tomograph. After being positioned in the scanner, a five minute transmission scan is obtained in order to correct for photon attenuation, and to check the positioning of the animal. The monkeys are then injected with 10-15 mCi of the IL-10 tracer, 6-[¹⁸F] fluoro-L-m-tyrosine (FMT) and imaging is begun. Imaging continues for 60 min, at which time the monkey is repositioned so as to image a second slice 6 mm caudal to the first.

The PET and MR datasets are co-registered and regions of interest (ROs) are drawn for the striatum in the contralateral hemisphere (the side opposite to ICA MPTP infusion) on PET data collected at 50 to 60 min (slice 1) and from 65 to 75 min (slice 2) with reference to the MR. Mirror images of the ROs are created in the ipsilateral hemisphere (side of MPTP infusion) and radioactivity counts (cm²/sec) are determined for each ROI. Striatal counts are averaged over the two slices for each study. FMT uptake asymmetry ratios are calculated for each animal at each time point by subtracting the counts for the ipsilateral (lesioned) striatum from the counts for the contralateral (un-lesioned) striatum and dividing by the average counts for the ipsilateral and contralateral striata. In order to reduce between animal variability in asymmetry ratios, a change score is calculated by subtracting the asymmetry ratio from the second PET study from the asymmetry ratio for the baseline study for each animal. Unpaired t-tests are used to compare the change in pet asymmetry ratios for the AAV-IL-10 and AAV-LacZ monkeys.

Necropsy

Animals are deeply anesthetized with sodium pentobarbital (25 mg/kg i.v.) and sacrificed 8-9 weeks following AAV administration and one week following postsurgical PET scans. On the day of sacrifice, blood samples are taken, and the animals are treated with L-dopa/carbidopa preparation (Sinemet 250/25). Plasma and cervical CSF are collected and at the time of necropsy. The brains are removed 30-45 minutes following the Sinemet administration, placed in the brain matrix and sectioned coronally into 3-6 mm slices. One 3 mm thick striatal brain slice from each monkey is immediately frozen in −70° C. isopentane and stored frozen for biochemical analysis. The remaining 6 mm thick slices are post-fixed in formalin for 72 hours, washed in PBS for 12 hrs and adjusted in ascending sucrose gradient (10-20-30%) and frozen.

Histological Analysis

The formalin-fixed brain slices are cut into 30 μm thick coronal sections in a cryostat. Frozen sections are collected in series starting at the level of the rostral tip of the caudate nucleus all the way caudally to the level of the substantia nigra. Each section is saved and kept in antifreeze solution at 70° C. Serial sections are stained for hIL-10 immunoreactivity (IR). Every 12th section is washed in phosphate buffered saline (PBS) and incubated in 3% H₂O₂ for 20 min to block the endogenous peroxidase activity. After washing in PBS, the sections are incubated in blocking solution for 30 min, followed by incubation in a solution comprising anti-hIL-10 antibody (rabbit monoclonal, 1:1000, Chemicon, Temecula, Calif.) for 24 h. The sections are then incubated for 1 h in biotinylated anti-rabbit IgG secondary antibody (1:300, Vector Labs, Burlingame, Calif.). The antibody binding is visualized with streptavidin horseradish peroxidase (Vector Labs, 1:300) and DAB chromogen with nickel (Vector Labs). Sections are then coverslipped and examined under a light microscope. Following tissue punching the fresh-frozen blocks are sectioned at 20 μm. Sections are stained with H&E and for hIL-10-IR.

Quantitative estimates of the total number of AAV-infected cells within the caudate nucleus, putamen and globus pallidus are determined using an optical dissector procedure. The optical dissector system consists of a computer assisted image analysis system, including an Leitz Ortholux 11 microscope hard-coupled to a Prior H128 computer-controlled x-y-z motorized stage, a high sensitivity Sony 3CCD video camera system (Sony, Japan) and a Macintosh G-3 computer. All analyses are performed using NeuroZoom software (Neurome, La Jolla, Calif.). Prior to each series of measurements, the instrument is calibrated. The region of positive neurons in the caudate, putamen and globus pallidus is outlined at low magnification (2.5× objective). If there is a diffuse presence of AAV-infected cells within the striatum, 1% of the outlined region is measured with a systematic random design of dissector counting frames (1 505 1IM2) using a 63× plan-Neofluar® immersion objective with a 0.95 numerical aperture. At least four sections equally spaced are sampled.

Once the top of the section is in focus, the z-plane is lowered a 1-2 gm. Counts are than made while focusing down through three 5 μm-thick dissectors. Care must be taken to ensure that the bottom forbidden plane is never included in the analysis. The volumes of the structures are calculated according to standard procedures. The total number of positive cells in the examined structures is calculated by using the formula N=Nv×Vs, where Nv is the numerical density and Vs is the volume of the structure.

Areas adjacent to cannula tracts may be stained with Nissi and H&E staining. GFAP-immunostaining may also be performed.

### EXAMPLE 6

### Treatment of Parkinson's Disease Using Recombinant AAV Virions Encoding IL-10

Viral vector AAV-hIL-10 is prepared as disclosed in Examples 1 and 2. Parkinson's patients are bilaterally infused with a total volume of 200-600 μL spread over four sites (two sites in left putamen, two sites in right putamen; 50-150 μL per site), with a total dose of 9×10¹⁰ to 9×10¹⁵ vg/subject. AAV-hIL-10 is administered to the striatum by intrastriatal infusion delivered by means of a stereotactically positioned cannula. The administration device includes a surgical stainless steel cannula with a stepped design to facilitate convection enhanced delivery, biocompatible Teflon tubing, and a syringe. The device is attached to a syringe pump to achieve a consistent rate of infusion of 1-3 μL per minute.

Post-surgical visits occur at 1, 2 and 4 weeks post-surgery. The visits at weeks 1 and 2 primarily involve post-surgical care, e.g. dressing change. Subjects are followed for a total of 6 months, with examinations occurring at 1-month intervals until the third month. The subject undergoes FMT-PET scans at one and six months post-surgery to assess IL-10 expression level. Behavioral assessments will occur at baseline, three and six months.

While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

All references cited herein, including without limitation, patents, patent application publications, journal articles, books and database entries, are hereby incorporated by reference in their entireties regardless of whether they are specifically incorporated elsewhere in this application.

What is claimed is:

1. A method for treating a neurodegenerative disease in a subject, said method comprising:
(a) providing a preparation of recombinant adeno-associated virus (rAAV) vectors comprising a nucleic acid sequence encoding an anti-inflammatory cytokine; and
(b) delivering the preparation to the central nervous system (CNS) of the subject, whereby said vectors transduce one or more cells in the CNS, and
whereby the anti-inflammatory cytokine sequence is expressed by the transduced cells at a therapeutically effective level.

2. The method of claim 1, wherein the anti-inflammatory cytokine is IL-10.

3. The method of claim 1, wherein the neurodegenerative disease is Parkinson's disease.

4. The method of claim 1, wherein the rAAV vector is provided as a plasmid.

5. The method of claim 1, wherein the rAAV vector is provided as rAAV virions.

6. The method of claim 1, wherein said preparation is delivered by intranasal delivery.

7. The method of claim 1, wherein said preparation is delivered intrathecally.

8. The method of claim 1, wherein said delivering is to the dorsal root ganglion (DRG).

9. The method of claim 1, wherein said delivering is to the brain.

10. The method of claim 9, wherein said delivering is to the striatum.

11. The method of claim 9, wherein said delivering is to the substantia nigra.

12. The method of claim 1, wherein said delivering is by convection enhanced delivery (CED).

13. A method for treating a neurodegenerative disease in a subject, said method comprising:
(a) providing a preparation of an anti-inflammatory cytokine; and
(b) delivering the preparation to the central nervous system (CNS) of the subject,
whereby the anti-inflammatory cytokine achieves a therapeutically effective level in the CNS.

14. The method of claim 13, wherein the anti-inflammatory cytokine is IL-10.

15. The method of claim 13, wherein the neurodegenerative disease is Parkinson's disease.

16. The method of claim 13, wherein said preparation is delivered by intranasal delivery.

17. The method of claim 13, wherein said preparation is delivered intrathecally.

18. The method of claim 13, wherein said delivering is to the dorsal root ganglion (DRG).

19. The method of claim 13, wherein said delivering is to the brain.

20. The method of claim 19, wherein said delivering is to the striatum.

21. The method of claim 19, wherein said delivering is to the substantia nigra.

22. The method of claim 13, wherein said delivering is by convection enhanced delivery (CED).
